(19) United States
(12) Patent Application Publication
(10) Pub. No.: US 2019/0076535 A1
(43) Pub. Date: Mar. 14, 2019

(54) CROSSLINKED PROTEIN-POLYSACCHARIDE COMPLEXED CONSUMABLE MACROCAPSULE

(71) Applicants: Joshua Alan Held, Santa Monica, CA (US); Margaret Teresa Thomas Virgallito, Santa Monica, CA (US)

(72) Inventors: Joshua Alan Held, Santa Monica, CA (US); Margaret Teresa Thomas Virgallito, Santa Monica, CA (US)

(21) Appl. No.: 15/436,976

(22) Filed: Feb. 20, 2017

(51) Int. Cl. A61K 47/42; A61K 47/36; A61K 47/38

(52) U.S. Cl. CPC A61K 47/42; A61K 47/38; A61K 47/36

(57) ABSTRACT

A consumable macromolecular article of manufacture formed by the complexation of protein and polysaccharide polymers stabilized by chemical or physical crosslinking and subsequently cast and molded into macrocapsules of at least 1 mm in diameter. The resulting macrocapsules are a highly versatile delivery product capable of encapsulating and stably retaining a wide range of active ingredients, both liquids and solids, in the amorphous regions of the complexed and crosslinked macromolecular structure, and releasing such active ingredients only upon chewing and/or upon exposure to the digestive environment. Dissimilar layers may be utilized to combine multiple active ingredients and/or control the active ingredient release profiles. The article of manufacture is thermally stable and structurally robust providing relatively low-cost commercial manufacture and distribution and exhibiting a texture and consistency that is organolepically pleasing.

### FIELD OF THE PRESENT DISCLOSURE

This disclosure relates generally to innovative chewable delivery products and more particularly an innovative crosslinked protein-polysaccharide complexed consumable macrocapsule for delivering a wide range of active ingredients.

### BACKGROUND OF THE RELATED ART

There exists a large and competitive market for consumable delivery products for various active ingredients such as dietary nutriments and supplements, nutraceuticals, and pharmaceuticals. Some legacy delivery products include dehydrated powders, pills, chewable pills, gel capsules, and protein or polysaccharide based gels. To be successful, a delivery product must be capable of stably retaining and preserving the particular desired active ingredients until consumption and also must be capable of presenting such active ingredients for ingestion in an appetizing, chewable, and organoleptically pleasing manner.

Because the market is very competitive, it is preferable that a delivery product is as convenient and versatile as possible, possessing a minimum of negative qualities such as storage and distribution inconveniences, and also be innovative and interesting in a manner that peaks the interests of consumers. In this regard, each of the above-mentioned legacy delivery products are all limited or fall short in some capacity.

For example, while dehydrated powders may be convenient in terms of storing large quantities of active ingredients in a compact and lightweight manner, powders typically must be hydrated before consumption; therefore, when delivering active ingredients through powders some minimal preparation effort before consumption and some minimal clean-up effort after consumption is necessary, making the powder based delivery platform products less convenient and therefore less attractive to some individuals than ready-to-consume options.

Pills and gel capsules are examples of ready-to-consume options that are compact and light-weight providing for convenient storage and transportation. However, there are some individuals who have difficulty with swallowing pills and because of the size limitations of pills the quantity of active ingredient delivered in each pill is limited. There are also many others who simply do not enjoy swallowing pills. For these such individuals, dry chewable pills are a slightly more attractive alternative. However, dry chewable pills are limited as well because chewable pills are usually reserved for delivering dry active ingredients and may not provide the best organoleptic experience, especially if the active ingredients have a less than pleasant taste.

Protein based or polysaccharide based gels, including consumables that are sometimes called sometimes called gummies, are examples of ready-to-consume options that can provide a more pleasant consumption experience because they are typically soft and may be chewed ease. Unlike dry chewable pills, gummies and other protein or polysaccharide based gels do not typically crumble when bitten allowing such consumables to be slightly larger in size because they can be conveniently consumed in two or more separate bites. The larger sizes allow each consumable to potentially deliver an increased quantity of active ingredient.

Legacy protein or polysaccharide based gels and gummies have significant limitations as well. For instance, the process of making gummy products can be detrimental to the active ingredients they are intended to delivery. In some cases, the finished gummy product may contain as little as half of the active ingredient that was originally introduced into the manufacturing process. This can be a substantial economic disadvantage from a manufacturing point of view, especially when delivering relatively expensive active ingredients.

Legacy gels and gummies are also not typically very structurally strong. In fact, some gels are not even designed to be self-contained such as the polysaccharide based gel disclosed in U.S. Pat. No. 9,414,615 which is a gel that must be stored and distributed inside some sort of structural shell or packaging. Similarly, energy gels designed to replenish sugars and nutrients during exercise are comprised of simple and complex carbohydrates and have no structural integrity whatsoever and must packaged in a pouch or container.

Other protein based or polysaccharide based products have structural integrity at rest but will fail under even the slightest compressive force making commercial manufacture and distribution of such consumables extremely challenging.

Structural integrity tends to further deteriorate with temperature. Many such protein or polysaccharide based gels and gummies are thermally reversible and will begin to melt or seep fluids if they warm to a particular critical temperature which varies depending on the particular protein or polysaccharide and the nature of the other ingredients but, in some cases, can be as low as room temperature (approximately 22 degrees Celsius).

In some cases refrigeration might be necessary to keep such consumables in a solid state, the commercial distribution of which may be extra expensive and challenging because of the need to keep such products relatively cool from the time of manufacture to until consumption. There exists a need for a gel or gummy based delivery product that is both structurally and thermally stable so that it may be commercially manufactured and distributed without requiring special handling.

Another limited legacy article of manufacture involving proteins and polysaccharides is the microcapsule. The microcapsule is constructed from the combination of protein and polysaccharide polymers and is an improvement over protein only based gels and polysaccharide only gels because the microcapsule is comprised of both protein and polysaccharide polymers which typically increases the structural strength of the consumable.

Microcapsules are manufactured in situ from the hydrocolloid of coacervatated protein and polysaccharide polymers that seed or nucleate around emulsified oil droplets triggered by specific and purposeful temperature and pH manipulations. The method of manufacture and physical structure of microcapsules are disclosed in much greater detail in U.S. Pat. Nos. 6,039,901 and 6,325,951.

While microcapsules can be used to encapsulate and deliver a wide variety of active ingredients, this process is only capable of manufacturing capsules in the 10-300 micron range which is much too small to serve as an attractive delivery product for many applications. Microcapsules can, however, be incorporated into larger delivery products to assist in the delivery profile of the active ingredients.

There exists a strong demand for an attractively sized chewable consumable delivery product for presenting various active ingredients, that exhibits both structural and thermal integrity within acceptable force and temperature ranges to accommodate standard commercial manufacture and distribution, and that also can provide a pleasing organoleptic experience.

The present disclosure distinguishes over the related art providing heretofore unknown advantages as described in the following summary.

### BRIEF SUMMARY OF THE INVENTION

The present disclosure describes a complexed and crosslinked protein-polysaccharide complexed consumable macrocapsule capable of encapsulating and delivering a wide range of active ingredients, both liquid and solid, for consumption innovative an chewable consumable.

The macrocapsule is not limited in size by its manufacturing process like the previously described microcapsule because rather than being formed in situ by seeding or nucleation oil particles, when forming the macrocapsules the entire complexed hydrocolloid comprised of a protein polymer and polysaccharide polymer and other ingredients is cast into molds that dictate the macrocapsule's ultimate size. This process allows for the creation of much larger structures than possible through the microcapsule manufacturing process. The typical size range of a macrocapsule is 1 to 300 mm, however, larger macrocapsules are possible because the size of the macrocapsule is dictated primarily by the size of the mold.

The resulting macrocapsule is a gel-like pleasurable texture which is soft and chewable and exhibits an improved structural and thermal integrity over legacy protein or polysaccharide gels and/or gummies. The increased physical robustness is due to both the advantages of complexing a protein polymer with a polysaccharide polymer and the subsequent crosslinking which strengthens the resulting complexed polymers.

Whether due to the introduction of a chemical crosslinker or through physical crosslinking induced by temperature or pH manipulation, crosslinking fortifies both the structural and thermal integrity of the consumable macrocapsule. Once the crosslinking has occurred the polymer complex is irreversible and therefore more robust. With increased structural integrity and thermal integrity over legacy protein and polysaccharide based gels, the presently disclosed crosslinked protein-polysaccharide complexed consumable macrocapsule is much more compatible with the requirements and limitations of commercial manufacture and distribution.

The presently disclosed consumable is capable of encapsulating and delivering a wide range of active ingredients, both liquid and solid, and delivering such active ingredients for a pleasurable consumption experience. The active ingredients are encapsulated in the amorphous regions of the crosslinked protein-polysaccharide complex polymeric structure. The retention and release of the encapsulated material is superior to other gel systems in that the encapsulated active ingredients are only released upon chewing or exposure to the environment of the oral cavity. This increases the shelf life of the delivery products and serves the added advantage of minimizing any unwanted interactions between multiple encapsulated active ingredients.

The presently disclosed crosslinked protein-polysaccharide consumable is created by complexing a water soluble protein with a water soluble polysaccharide in water with a pH between 4 and 7 solution at a temperature between 40 to 75 degrees Celsius. When mixed with the proper vigor, both the protein and the polysaccharide and begin to coacervate. While in solution, macromolecular hydrogel colloids develop as the result of the formation of linkages between the protein and the monomeric constituents of the polysaccharide, with the protein being the cationic polymer and the polysaccharide being the anionic polymer.

There is a wide variety of acceptable water-soluble proteins at may be utilized. Each different protein in combination with each different polysaccharide has the potential to form a macrocapsule with a slightly different organoleptic feel. Some preferred proteins include: porcine gelatin with a bloom strength of 200-300 bloom; collagen; keratin; myofibrillar protein; whey protein concentrate; whey protein isolate; whey protein hydrolysate; native whey, native whey concentrate; native whey isolate, casein, ovalbumin, ovomucoid, ovoglobulin, conalbumin, vitellin, vitellenin, legumin, albumin, and any combination thereof.

Some preferred polysaccharides include: pectin; agar; iota carrageenan; kappa carrageenan gum; gum arabic; sucrose; pullulan; guar gum; locust bean gum; chitosan; hydroxyl propyl methyl cellulose; carboxyl methyl cellulose; cellulose; konjac gum; gellan gum; dextran; dextrin; glucose, chitin; tragacanth gum; karaya gum; tara gum; carob; fenugreek; beta-mannan; galactomannans; beta glucan; sodium alginate; inulin, poly dextrose; hemi cellulose; glycogen; starch; modified starch; maltodextrin; galactan; isolichen; laminaran; lavans; yeast mannan; and any combination thereof.

The listed preferred proteins and polysaccharide are not mean to be limiting as there exists other unremunerated proteins and polysaccharides that are acceptable and possibly even preferable. The important quality is the manner in which the chosen protein polymer complexes with the chosen polysaccharide. If the polymers complex well then the polymers will likely make a very good consumable macrocapsule.

Once the protein polymer and polysaccharide polymer are full complexed and forms a hydrocolloid, the polymers are crosslinked and cast in molds and allowed to fully gel. The crosslinking step may be initiated by adding a chemical crosslinker or it may be achieved through physical crosslinking achieved through manipulation of temperature, pressure, and pH.

Preferred chemical crosslinkers include: transglutaminase; genitipin; glutaraldehyde; tannic acid (tannin); potassium aluminum sulfate, sodium sulfate polyamines; oxidized dextrins; hydrazides; alkoxyamines; ketones; periodic acid; calcium chloride; calcium carbonate; calcium citrate; potassium citrate, and any combination thereof. As with the cited protein and polysaccaride polymers, the suggested chemical crosslinkers are not intended to be an exhaustive list and other crosslinkers exist that that may effectively crosslink the complexed polymers to achieve the same end.

The resulting protein-polysaccharide complex is cast into molds and allowed to set and gel. A major advantage of this article of manufacture is that virtually all the initial ingredients survive the manufacturing process and are present in the end product. This distinguished this process from protein-polysaccharide films that begin with a similar slurry of ingredients but evaporate the majority of the liquid off to form the finished product. Such products are limited in that they are incapable of encapsulating and delivering a liquid active ingredient.

There are several proteins and polysaccharide that may be utilized in creating different embodiments of the presently disclosed macrocapsule and by manipulating the ratios of ingredients the qualities of the resulting macrocapsule can be altered. However, there certain ingredient ratios that, if followed, ensure superior thermal and physical characteristic. Those ratios are the following: between 0.5% and 25% by weight said water-soluble protein; between 0.5% and 40% by weight said water-soluble polysaccharide; and between 0.5% and 90% by weight said active ingredient. When following these guidelines the resulting products should be capable of withstanding between to 5 and 10 Newton of compressive force and remain stability up to at least 60 degrees Celsius. The minimum compressive strength threshold is to ensure the microcapsule will not fail or disintegrate during commercial distribution and the upper threshold is design to maintain an appropriate softness for the organoleptic pleasure. With regard to temperature, the primary concern is that the microcapsule will not melt or become sticky during shipping or if exposed to a moderate hot day. A macrocapsule that does not need special handling or attention is more valuable as a delivery system both during and after commercial distribution.

Extensive testing was conducted to arrive at the ingredient ratios set forth above. The following are the results of a few exemplar arrays demonstrating the advantages of including both a protein and a polysaccharide polymer, and the advantage of including a crosslinker. The compression testing was conducted using a SHIMPO FGV 10X.

In example array 1, formula 1 is a pure protein polymer without the additional of a crosslinker. The resulting microcapsule exhibited an ideal structural integrity with an relatively low melting point. When a crosslinker was added in formula 2, the melting point was increased to an acceptable level but the breaking strength increased to a level that may exceeds the pleasurable tastes of many people. In formula 3, when the protein and polysaccharide polymers where mixed at a 50:50 ratio, both the breaking strength and the melting point were unacceptably low. In formula 4, the 50:50 ratio of protein and polysaccharide used with the addition of a crosslinker and the breaking strength and melting point where within the preferable range.

In example array 2, both formula 1 and formula 2 used a 50:50 ratio of protein and polysaccharide polymers but only formula 2 included a crosslinker. Once again, the breaking strength of the macrocapsule without the crosslinker demonstrated insufficient strength.

In example array 3, a 50:50 mixtures of protein and polysaccharaide was used with chicken gelatin protein. A crosslinker was added to only formula 2. As expected, formula 1 exhibited an insufficient break strength and melting point, whereas, with a crosslinker included, the compression break strength was an ideal 8.4 N and the melting point rose to 63° Celcius.

This disclosure teaches certain benefits in construction and use which give rise to the objectives described below.

A primary objective inherent in the above disclosure is to provide a crosslinked protein-polysaccharide macromolecular complexed consumable macrocapsule for delivering a wide range of encapsulated active ingredients.

Another objective of the above disclosure is to provide a crosslinked protein-polysaccharide macromolecular complexed consumable and thermally stable macrocapsule for delivering a wide range of encapsulated active ingredients

A further objective of the above disclosure is to provide a crosslinked protein-polysaccharide macromolecular complexed consumable and more structurally robust macrocapsule for delivering a wide range of encapsulated active ingredients.

A still further objective of the above disclosure is to provide a crosslinked protein-polysaccharide macromolecular complexed consumable macrocapsule for delivering a wide range of encapsulated sold and liquid state active ingredients.

A yet still further objective of the above disclosure is to provide a crosslinked protein-polysaccharide macromolecular complexed macrocapsule for delivering a wide range of encapsulated active ingredients with minimal negative organoleptic qualities.

A yet still further objective of the above disclosure is to provide a thermally stable crosslinked protein-polysaccharide macromolecular complexed consumable macrocapsule for delivering a wide range of encapsulated active ingredients with a variety of release profiles.

Other features and advantages of the present invention will become apparent from the following more detailed description, taken in conjunction with the accompanying drawings, which illustrate, by way of example, an exemplary embodiment of the presently described article of manufacture.

### BRIEF DESCRIPTION OF THE SEVERAL VIEWS OF THE DRAWING(S)

The accompanying drawings illustrate exemplary implementations and are part of the specification. The illustrated implementations are proffered for purposes of example not for purposes of limitation. Illustrated elements will be designated by numbers. Once designated, an element will be identified by the identical number throughout. Illustrated in the accompanying drawings in at least one of the best mode embodiments of the present disclosure. In such drawings:

FIG. 1 is an perspective view of an exemplary embodiment of a crosslinked protein-polysaccharide complexed consumable macrocapsule.

FIG. 2 is a perspective view of a cross section of an exemplary embodiment of a crosslinked protein-polysaccharide complexed consumable macrocapsule.

FIG. 3 is a perspective view of an exemplary embodiment of a crosslinked protein-polysaccharide complexed consumable macrocapsule that is depicted in the grasp of an index finger and thumb of a human hand for size context.

FIG. 4A is a perspective view of a cross section of an exemplary embodiment of a crosslinked protein-polysaccharide complexed consumable macrocapsule illustrating a homogenous distribution of the encapsulated active ingredient.

FIG. 4B is a plan view of a cross section of an exemplary embodiment of a crosslinked protein-polysaccharide complexed consumable macrocapsule illustrating a homogenous distribution of the encapsulated active ingredient.

FIG. 5A is a perspective view of a cross section of an exemplary embodiment of a crosslinked protein-polysaccharide complexed consumable macrocapsule illustrating distribution of heterogeneous concentrations of encapsulated active ingredient.

FIG. 5B is a plan view of a cross section of an exemplary embodiment of a crosslinked protein-polysaccharide complexed consumable macrocapsule illustrating a distribution of heterogeneous concentrations of encapsulated active ingredient.

FIG. 6A is a perspective view of a cross section of an exemplary embodiment of a crosslinked protein-polysaccharide complexed consumable macrocapsule illustrating distribution of heterogeneous concentrations of encapsulated active ingredient surrounded by a dissimilar exterior layer for the purposes of controlling the release profile.

FIG. 6B is a plan view of a cross section of an exemplary embodiment of a crosslinked protein-polysaccharide complexed consumable macrocapsule illustrating distribution of heterogeneous concentrations of encapsulated active ingredient surrounded by a dissimilar exterior layer for the purposes of controlling the release profile.

FIG. 7A is a perspective view of a cross section of an exemplary embodiment of a crosslinked protein-polysaccharide complexed consumable macrocapsule illustrating the homogeneous distribution of the encapsulated active ingredient surrounded by multiple dissimilar exterior layers for the purposes of controlling the release profile.

FIG. 7B is a plan view of a cross section of an exemplary embodiment of a crosslinked protein-polysaccharide complexed consumable macrocapsule illustrating the homogeneous distribution of the encapsulated active ingredient surrounded by multiple dissimilar exterior layers for the purposes of controlling the release profile.

### DETAILED DESCRIPTION OF THE EXEMPLARY EMBODIMENT'S DEPICTED IN THE DRAWINGS

The drawing figures illustrate various exemplary embodiments of the crosslinked protein-polysaccharide complexed consumable article of manufacture in at least one of its preferred, best mode embodiments, which is further defined in detail in the following description. Those having ordinary skill in the art may be able to make alterations and modifications to what is described herein without departing from the spirit and scope of the disclosure. Further, it must be understood that what is illustrated is set forth only for the purposes of example and that it should not be taken as a limitation in the scope of the presently described article of manufacture.

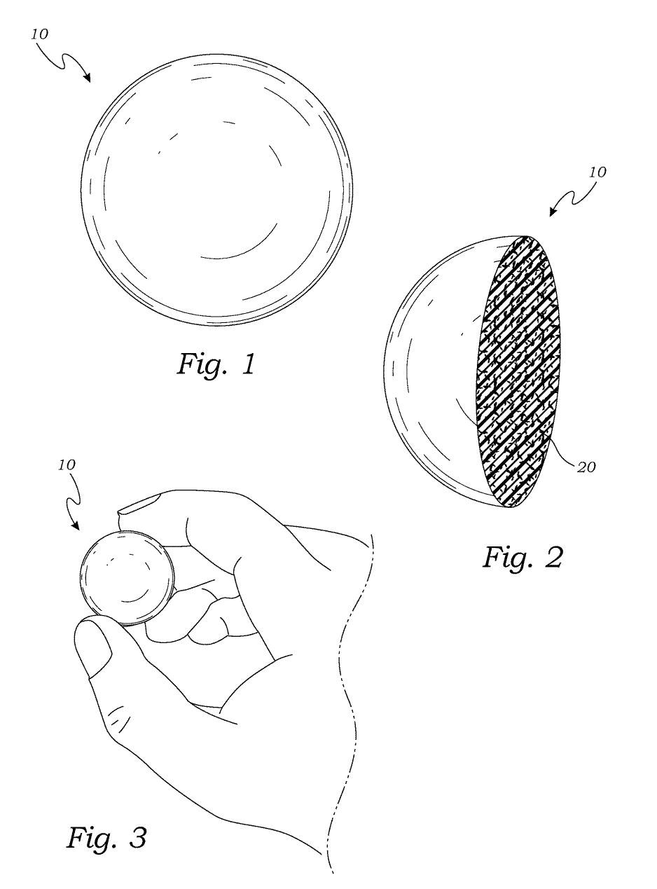

Illustrated in FIG. 1. is a novel and innovative crosslinked protein-polysaccharide complexed consumable macrocapsule 10 capable of encapsulating a wide variety of active ingredients. The article of manufacture 10 is able to exhibit a desired blend of physical, thermal, and organoleptic properties because of the unique individual properties of both the protein and polysaccharide subparts and because of the complex and synergistic manner in which they are combined and made irreversible by crosslinking.

FIG. 2 illustrates a cross section of the same macrocapsule 10 showing that the active ingredient is homogeneously distributed throughout the macrocapsule 10. The illustration includes a flake-like pattern 20 representing the possibility of a non-liquid active ingredient homogeneously distributed throughout the macrocapsule 10.

In FIG. 3 illustrates a cross section of a macrocapsule 10 being held between an index finger and a thumb. This illustration is not intended to be limiting. As previously stated and claimed, macrocapsules 10 can exhibit a range of sizes greater than 1 mm in diameter. A typical microcapsule 10 will have a diameter between 1 and 300 mm. Further, because macrocapsules 10 are cast is molds, they do not have to be spherical. Macrocapsules 10 can be formed in any shape that can be created by a mold. When referencing nonspherical macrocapsules 10, any reference to diameter refers to the largest cross sectional length.

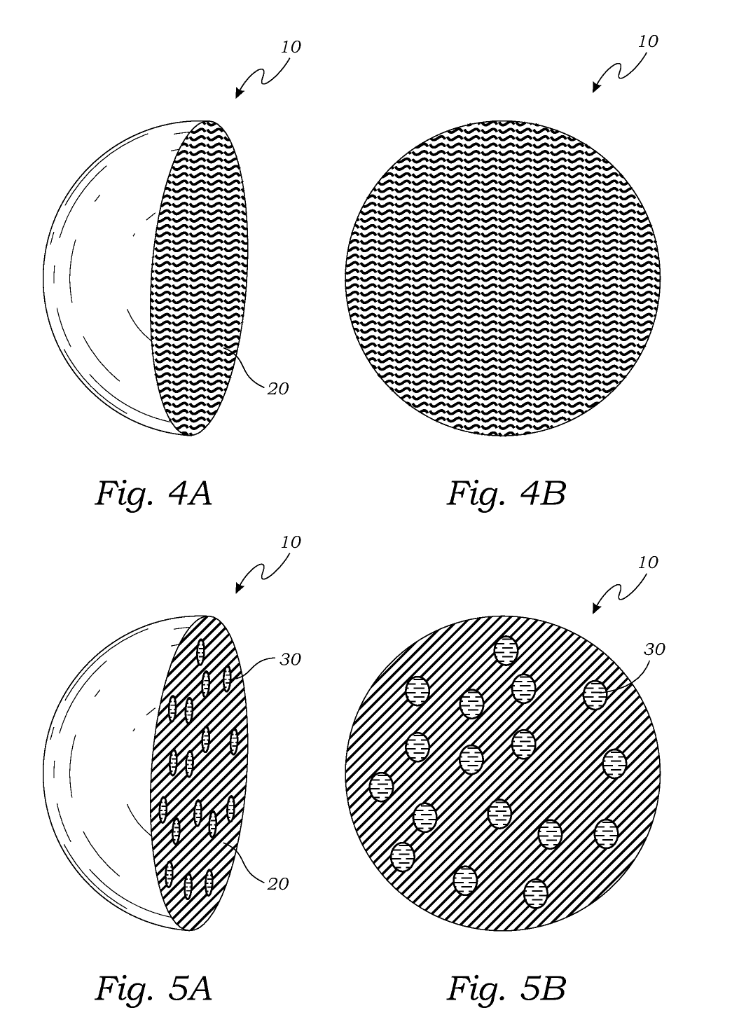

FIGS. 4A and 4B illustrates a cross section of a macrocapsule 10 showing that the active ingredient is homogeneously distributed 20 throughout the macrocapsule 10 without the flake-like pattern illustrated in FIG. 2. This illustration depicts either a soluble active ingredient or a liquid active ingredient. The presently disclosed crosslinked protein polysaccharide macrocapsule 10 is capable of encapsulating both liquid and solid active ingredients in the amorphous regions of the complexed and crosslinked polymers that form the macrocapsule 10.

FIGS. 5A and 5B both illustrate a cross sections of an exemplary macrocapsule 10 showing active ingredients heterogeneously distributed throughout in inclusions 30. The inclusions 30 could represent the only active ingredient that the macrocapsule 10 is delivering or they could be in additional to other active ingredients that are homogeneously distributed throughout the macrocapule 30. In some embodiments, the inclusions 30 could be microcapsules 10 used to control the release profile of the active ingredients.

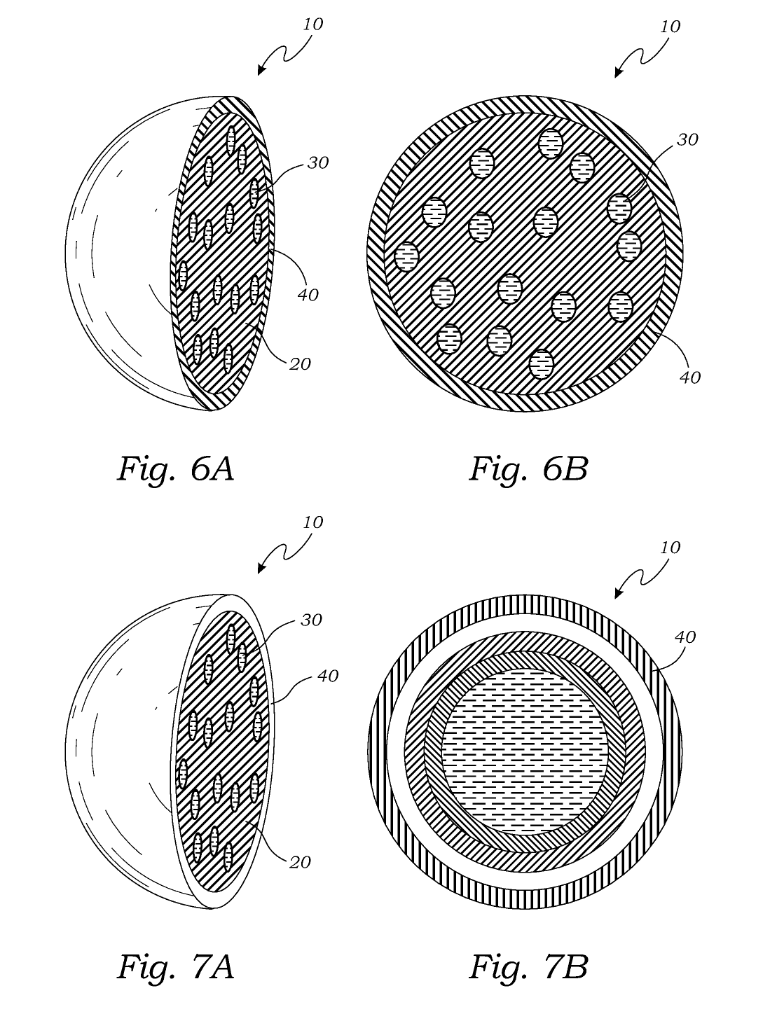

FIGS. 6A and 6B both illustrate a cross sections of an exemplary macrocapsule 10 similar to the embodiment illustrated the FIGS. 5A and 5B except that it further includes a distinctly separate external layer 40 that may contain a different active ingredients and/or different release profiles. In some embodiments, there may be multiple layers 40 with multiple different active ingredients and/or release profiles such as illustrated in FIG. 7. In other embodiments, an outer coating 40 may contain no active ingredient or release profile an serve the function to protect the macrocapsule 10 from the external environment.

The enablements described in detail above are considered novel over the prior art of record and are considered critical to the operation of at least one aspect of the presently described article of manufacture, and to the achievement of the above-described objectives. The words used in this specification to describe the instant embodiments are to be understood not only in the sense of their commonly defined meanings, but to include by special definition in this specification: structure, material, or acts beyond the scope of the commonly defined meanings. Thus, if an element can be understood in the context of this specification as including more than one meaning, then its use must be understood as being generic to all possible meanings supported by the specification and by the word(s) describing the element.

The definitions of the words or drawing elements described herein are meant to include not only the combination of elements which are literally set forth, but all equivalent structures, materials or acts for performing substantially the same function in substantially the same way to obtain substantially the same result. In this sense, it is therefore contemplated that an equivalent substitution of two or more elements may be made for any one of the elements described and its various embodiments or that a single element may be substituted for two or more elements in a claim.

Changes from the claimed subject matter as viewed by a person with ordinary skill in the art, now known or later devised, are expressly contemplated as being equivalents within the scope intended and its various embodiments. Therefore, substitutions, now or later known to one with ordinary skill in the art, are defined to be within the scope of the defined elements. This disclosure is thus meant to be understood to include what is specifically illustrated and described above, what is conceptually equivalent, what can be obviously substituted, and also what incorporates the essential ideas.

The scope of this description is to be interpreted only in conjunction with the appended claims and it is made clear, here, that the named inventors believe that the claimed subject matter is what is intended to be patented

What is claimed is:

1. An innovative article of manufacture for the consumption of a variety of active ingredients, said article of manufacture apparatus comprising:
a water-soluble protein;
a water-soluble polysaccharide;
an active ingredient; and
an chemical crosslinker, wherein said water-soluble protein and water-soluble polysaccharide are complexed, chemical crosslinked by said chemical crosslinker, and cast into molds to form macrocapsules which retain at least 95 percent of all ingredients by weight.

2. An article of manufacture as in claim 1 in which said macrocapsules are greater than 1 mm in diameter.

3. An article of manufacture as in claim 2 in which said macrocapsules are between 1 mm and 300 mm in diameter.

4. An article of manufacture as in claim 1 in which said macrocapsules comprise:
between 0.5% and 25% by weight said water-soluble protein;
between 0.5% and 40% by weight said water-soluble polysaccharide; and
between 0.5% and 90% by weight said active ingredient.

5. An article of manufacture as in claim 1 wherein said macrocapsules demonstrate thermal stability up to temperature of 60 degrees Celsius.

6. An article of manufacture as in claim 1 wherein said macrocapsules demonstrate structural stability under at least 5 Newton of compressive force.

7. An article of manufacture of claim 1 in which the protein polysaccharide macromolecular complex is chemically or physically crosslinked.

8. An article of manufacture of claim 1 wherein the macromolecular complex are formed by chemical reactions that are initiated by changes in temperature, pressure, and/or pH.

9. An article of manufacture of claim 1 wherein said active ingredient may comprise either a solid, liquid, or solid dissolved in solvent and at least 95 percent of said active ingredient survives the manufacturing process and is present in the finished article of manufacture.

10. An innovative delivery platform for the consumption of a variety of active ingredients, said article of manufacture apparatus comprising:
between 0.5% and 25% by weight water-soluble protein;
between 0.5% and 40% by weight water-soluble polysaccharide;
between 0.5% and 90% by weight active ingredient; and
an chemical crosslinker, wherein said water-soluble protein and water-soluble polysaccharide are complexed and chemical crosslinked to form macrocapsules at least 1 mm in diameter

11. An article of manufacture as in claim 1 wherein said water soluble protein is a protein selected form the following group: porcine gelatin with a bloom strength of 200-300 bloom; collagen; keratin; myofibrillar protein; whey protein concentrate; whey protein isolate; whey protein hydrolysate; native whey, native whey concentrate; native whey isolate, casein, ovalbumin, ovomucoid, ovoglobulin, conalbumin, vitellin, vitellenin, legumin, albumin, and any combination thereof.

12. An article of manufacture as in claim 1 wherein said water soluble polysaccacaride is a polysaccharide selected form the following group: pectin; agar; iota carrageenan; kappa carrageenan gum; gum arabic; sucrose; pullulan; guar gum; locust bean gum; chitosan; hydroxyl propyl methyl cellulose; carboxyl methyl cellulose; cellulose; konjac gum; gellan gum; dextran; dextrin; glucose, chitin; tragacanth gum; karaya gum; tara gum; carob; fenugreek; beta-mannan; galactomannans; beta glucan; sodium alginate; inulin, poly dextrose; hemo cellulose; glycogrn; starch; modified starch; maltodextrin; galactan; isolichen; laminaran; lavans; yeast mannan; and any combination thereof.

13. An article of manufacture as in claim 1 wherein said chemical crosslinker is a crosslinker selected form the following group: transglutaminase; genipin; glutaraldehyde; tannic acid (tannin); potassium aluminum sulfate, sodium sulfate polyamines; oxidized dextrins; hydrazides; alkoxyamines; ketones; periodic acid; calcium chloride; calcium carbonate; calcium citrate; potassium citrate, and any combination thereof.

14. An innovative article of manufacture for the consumption of a variety of active ingredients, said article of manufacture apparatus comprising:
a water-soluble protein;
a water-soluble polysaccharide;
an active ingredient; and
wherein said water-soluble protein and water-soluble polysaccharide are complexed, physically crosslinked, and cast into molds to form macrocapsules that the finished article of manufacture retains at least 95 percent of all constituent ingredients by weight.

15. An article of manufacture as in claim 14 in which said macrocapsules are between 1 mm and 300 mm in diameter.

16. An article of manufacture as in claim 15 in which said macrocapsules are greater than 1 mm in diameter.

17. An article of manufacture as in claim 16 in which said macrocapsules comprise:
between 0.5% and 25% by weight said water-soluble protein;
between 0.5% and 40% by weight said water-soluble polysaccharide; and
between 0.5% and 90% by weight said active ingredient.

18. An article of manufacture as in claim 17 wherein said macrocapsules demonstrate thermal stability up to at least 50 degrees Celsius.

19. An article of manufacture as in claim 18 wherein said macrocapsules demonstrate structural stability under at least 5 Newtons of compressive force.
